(19) United States
(12) Patent Application Publication
(10) Pub. No.: US 2026/0130935 A1
(43) Pub. Date: May 14, 2026

(54) BOVINE SUPPLEMENTS FOR INCREASING ENERGY AND CALCIUM LEVELS

(71) Applicant: Jason Krause, Pleasant Grove, UT (US)

(72) Inventor: Jason Krause, Pleasant Grove, UT (US)

(21) Appl. No.: 19/388,985

(22) Filed: Nov. 13, 2025

Related U.S. Application Data
(60) Provisional application No. 63/720,006, filed on Nov. 13, 2024.

(51) Int. Cl. A61K 33/06; A61K 31/19; A61K 31/455; A61K 36/06; A61P 3/00

(52) U.S. Cl. CPC A61K 33/06; A61K 31/19; A61K 31/455; A61K 36/06; A61P 3/00

(57) ABSTRACT

This disclosure relates generally to compositions, and/or methods of use thereof regarding compositions for increasing energy and calcium levels in cows. In various aspects, a composition for increasing calcium levels in a cow includes calcium propionate provided in a concentration ranging from about 80% to about 88% by weight. The calcium propionate may be both a glucose precursor and a source of calcium to increase calcium levels in the cow. The composition may also include at least one probiotic component provided in a concentration ranging from about 6% to about 10% by weight and niacin provided in a concentration ranging from about 6% to about 10% by weight. The niacin may be a glucose precursor to increase energy levels in the cow. The composition may be administered orally.

### CROSS REFERENCE TO RELATED APPLICATION

This application is a continuation of U.S. patent application Ser. No. 63/720,006, filed Nov. 13, 2024, titled BOVINE SUPPLEMENTS FOR INCREASING ENERGY AND CALCIUM LEVELS (“the '006 Provisional Application”). The entire disclosures of the '006 Provisional Application is hereby incorporated herein.

### TECHNICAL FIELD

This disclosure relates generally to compositions for increasing energy and calcium levels in animals and more particularly in bovines or cows, which compositions may be administered orally.

### SUMMARY

Disclosed are systems, compositions, and/or methods of use thereof regarding compositions for increasing energy and calcium levels in animals and more particularly cows. In various aspects, a composition for increasing calcium levels in a cow includes calcium propionate provided in a concentration ranging from about 80% to about 88% by weight. The calcium propionate may be both a glucose precursor and a source of calcium to increase calcium levels in the cow. The composition may also include at least one probiotic component provided in a concentration ranging from about 6% to about 10% by weight and niacin provided in a concentration ranging from about 6% to about 10% by weight. The niacin may be a glucose precursor to increase energy levels in the cow.

In various aspects, a composition for increasing propionic acid production in a cow includes calcium propionate provided in a concentration ranging from about 80% to about 88% by weight. The calcium propionate may be for increasing propionic acid production in the cow. Additionally, the calcium propionate may be both a glucose precursor and a source of calcium to increase calcium levels in the cow. The composition may also include at least one probiotic component provided in a concentration ranging from about 6% to about 10% by weight. The at least one probiotic component may include a yeast. Additionally, the composition may include niacin provided in a concentration ranging from about 6% to about 10% by weight. The niacin may be a glucose precursor to increase energy levels in the cow.

In various aspects, a composition for administration to a cow may include calcium propionate provided in a concentration of about 86% by weight, with the calcium propionate being both a glucose precursor and a source of calcium to increase calcium levels in the cow. The composition may also include at least one probiotic component provided in a concentration of about 7% by weight and niacin provided in a concentration of about 7% by weight, with the niacin being a glucose precursor to increase energy levels in the cow.

In various aspects, a composition for administration to a cow may include calcium propionate provided in a concentration ranging from about 80% to about 88% by weight, with the calcium propionate being both a glucose precursor and a source of calcium to increase calcium levels in the cow. The composition may also include at least one probiotic component provided in a concentration ranging from about 6% to about 10% by weight and niacin provided in a concentration ranging from about 6% to about 10% by weight. The niacin may be a glucose precursor to increase energy levels in the cow. The calcium propionate and the niacin may be the only sources of glucose precursors in the composition.

In various aspects, a method of increasing energy and calcium levels in a cow includes administering to the cow a therapeutically effective amount of a composition for increasing energy and calcium levels. The composition may include calcium propionate provided in a concentration ranging from about 80% to about 88% by weight, with the calcium propionate being both a glucose precursor and a source of calcium to increase calcium levels in the cow. The composition may also include at least one probiotic component provided in a concentration ranging from about 6% to about 10% by weight and niacin provided in a concentration ranging from about 6% to about 10% by weight. The niacin may be a glucose precursor to increase energy levels in the cow.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIGS. 1A and 1B are flowcharts of example methods of treating cows, according to the present disclosure;

FIG. 1C is a flowchart of an example process for creating at least one bolus;

FIG. 2 shows in vivo production of volatile fatty acids and ammonium resulting from submergence of a sample containing propylene glycol in rumen fluid from a cow for about 5 hours;

FIG. 3 shows in vivo production of volatile fatty acids and ammonium resulting from submergence of a sample containing propylene glycol in rumen fluid from a cow four about 5 hours;

FIG. 4 shows in vivo production of volatile fatty acids and ammonium resulting from submergence of a sample containing keto gel in rumen fluid from a cow for about 24 hours;

FIG. 5 shows in vivo production of volatile fatty acids and ammonium resulting from submergence of a sample containing propylene glycol in rumen fluid from a cow four about 24 hours;

FIG. 6 shows in vivo production of volatile fatty acids and ammonium resulting from submergence of a sample containing keto gel in rumen fluid from a cow for about 24 hours;

FIG. 7 shows in vivo production of volatile fatty acids and ammonium resulting from submergence of a sample containing propylene glycol in rumen fluid from a cow four about 24 hours;

FIG. 8 shows in vivo production of volatile fatty acids and ammonium resulting from submergence of a sample containing the disclosed composition in rumen fluid from a cow four about 24 hours;

FIG. 9 shows in vivo production of volatile fatty acids and ammonium resulting from submergence of a sample containing the disclosed composition in rumen fluid from a cow four about 24 hours; and

FIG. 10 shows in vivo production of volatile fatty acids and ammonium resulting from submergence of a sample containing the disclosed composition in rumen fluid from a cow four about 24 hours.

### DETAILED DESCRIPTION

Cows who have just given birth (i.e., “fresh cows”) begin to produce milk to provide the new calf with nutrients. Milk production is an energetically expensive process. Cows are genetically predisposed to always give priority to milk production, even if this is at the expense of the cow's own energy and health. If the energy provided through feed and rations is not sufficient, the cow will compensate by using its own body reserves. This leads to a decrease in available energy stores for the cow, which further leads to decreases in the cow's health and well-being. Decreases in energy stores are typically accompanied by decreased calcium levels.

Additionally, when there is not enough available energy for the cow, the cow will start to mobilize fat stores to acquire the necessary energy for milk product. If an excess of fat mobilization occurs, then ketone bodies may appear and build up in the cow's blood. In limited amounts, these ketones don't present a problem, but when larger concentrations are produced (a condition known as ketosis) the cow will appear less active and their performance will start to suffer. Ketosis is the clinical expression of severe hyperketonemia and is a common disease of adult cattle. It typically occurs in dairy cows in early lactation and is most consistently characterized by hyporexia and decreased milk production.

Traditionally, propylene glycol is used to treat cows with low energy stores. However, propylene glycol only provides energy in the short term and it must be repeatedly administered. Further, propylene glycol does not provide the cow with any other beneficial components, such as calcium. Additional supplements (e.g., calcium vitamins, etc.) must be administered to the cow separately from the propylene glycol. Multiple administrations of both the propylene glycol and other supplements causes stress on the cow and is inefficient.

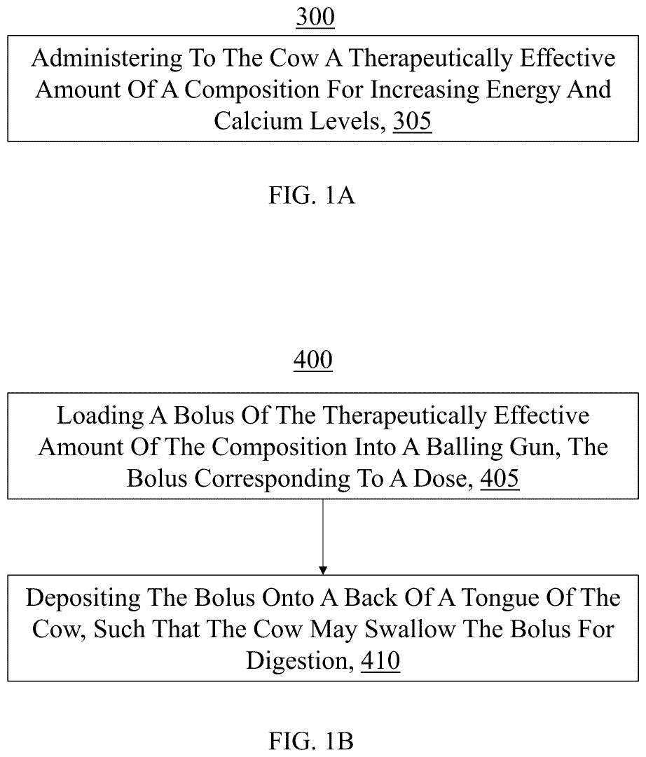

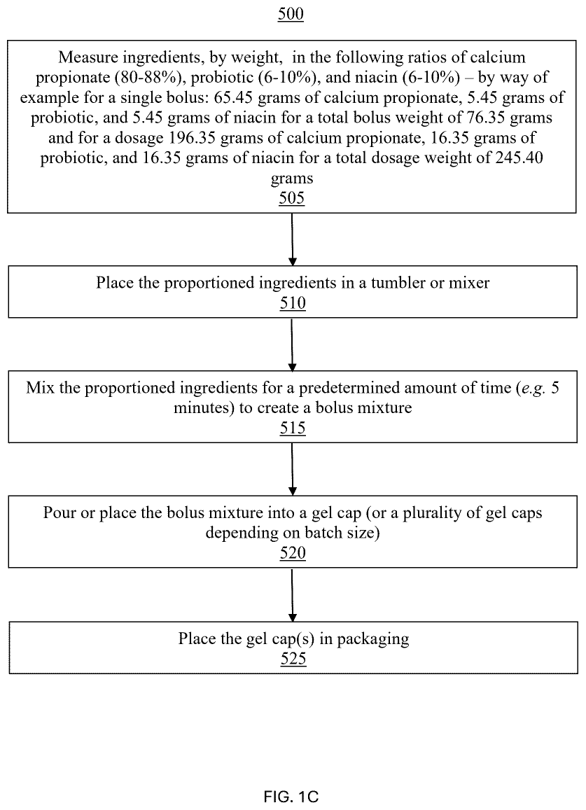

FIGS. 1A and 1B are flowcharts of example methods of treating cows, according to the present disclosure. FIG. 1A is a flowchart of an example method 300 of increasing energy and calcium levels in a cow. The method 300 includes administering to the cow a therapeutically effective amount of a composition for increasing energy and calcium levels, at 305. The composition may include calcium propionate, at least one probiotic component, and niacin.

The calcium propionate may be provided in a concentration ranging from about 80% to about 88% by weight. The calcium propionate may also be both a glucose precursor and a source of calcium to increase calcium levels in the cow. The at least one probiotic component provided in a concentration ranging from about 6% to about 10% by weight. In some embodiments, the at least one probiotic component includes a yeast or a combination of yeasts including, but not limited to saccharomyces cerevisiae. The niacin may be provided in a concentration ranging from about 6% to about 10% by weight and may be a glucose precursor to increase energy levels in the cow.

In some embodiments, administering to the cow a therapeutically effect amount of a composition may include administering a first dose of the composition to the cow at time one and administering a second dose of the composition to the cow at time two, with time two being later than/after time one. Administering to the cow a therapeutically effect amount of a composition may include administering three doses of the composition to the cow at once, such as loading three (3) boluses of the composition within a bolus gun, or balling gun, and depositing the boluses onto the back of the cow's tongue. The composition may be administered to a fresh cow, having given birth to a calf; a pre-fresh, pregnant cow; a ketonic cow; a sick cow; a cow going through metabolic challenges; or another condition appropriate for treatment with a composition containing calcium propionate, at least one probiotic component, and niacin.

FIG. 1B is a flowchart of a method 400 of increasing energy and calcium levels in a cow. The method 400 may include loading a bolus of the therapeutically effective amount of the composition into a bolus gun, the bolus corresponding to a dose, at 405. The method 400 may also include depositing the bolus onto a back of a tongue of the cow, such that the cow may swallow the bolus for digestion, at 410. The cow should have access to fresh water to help wash the bolus(es) down to the rumen of the cow for digestion. Ideally, the bolus(es) should not be administered to cows having defective swallow reflexes or to cows that are laying down, if possible.

FIG. 1C is a flowchart of an exemplary method 500 of making at least one, or a single, bolus to administer to a cow for the reasoning set forth herein. A single bolus may include the ingredients of the calcium propionate, the probiotic, and the niacin in the ratios as set forth herein. By example a single bolus may include 61.08 grams to 67.188 grams of calcium propionate, and between 4.581 grams to 7.635 grams of the probiotic, and 4.581 grams to 7.635 grams niacin for a total single bolus weight between 70.242 grams to 82.458 grams. A dosage to a cow is considered three boluses thus making the total dosage to a cow at any given time of calcium propionate between 183.24 grams to 201.564 grams, and of the probiotic between 13.743 grams to 22.905 grams, and the niacin between 13.743 grams to 22.905 grams.

The bolus(es) may be made in batches or may be made in a continuous mixing process. A bolus mixture is made by measuring out each ingredient 505 and positioned or placed in a mixer or tumbler 510. The ingredients are mixed for a relatively short period of time and may be as little as three minutes and as much as twenty minutes creating a bolus mixture 515. The mixture is then poured or placed into a dissolvable gel cap 520. The gel cap may be 110 milliliter gel cap for ease in administration with a bolus gun. The gel cap with the bolus mixture inside may be immediately packaged 525 or may set for a short period of time. The packaging may allow the boluses to be shipped in small or large quantities. It will be appreciated the large batches or continuous mixing processes may be created that proportionately create a single bolus and a dosage (three boluses).

Of the exemplary method set forth in FIG. 1C (by way of example for a single bolus: 65.45 grams of calcium propionate, 5.45 grams of probiotic, and 5.45 grams of niacin for a total bolus weight of 76.35 grams and for a dosage 196.35 grams of calcium propionate, 16.35 grams of probiotic, and 16.35 grams of niacin for a total dosage weight of 245.40) it will be appreciated that from dosage provided to the cow, the cow is able to utilize about, or at a minimum, 44 grams of available calcium, 185 grams of available glucose, 15 grams of probiotic and 15 grams of niacin.

### Examples and Methodology

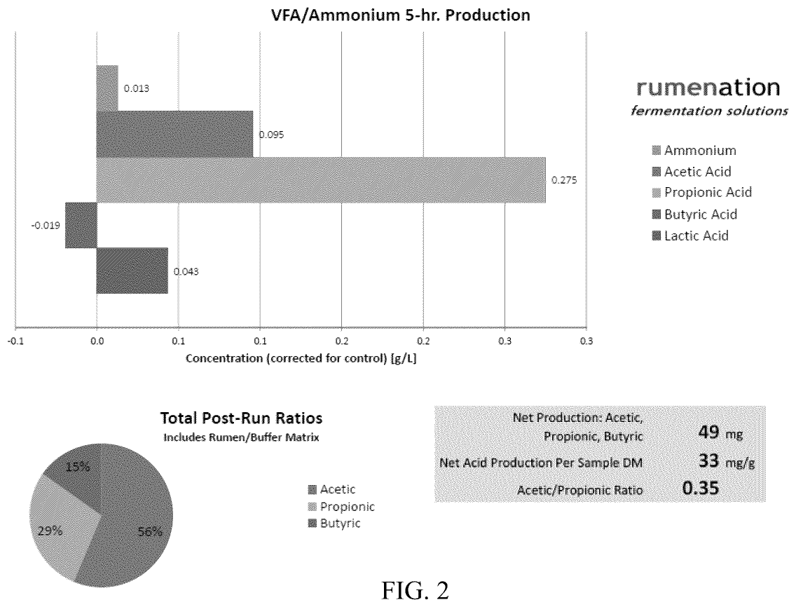

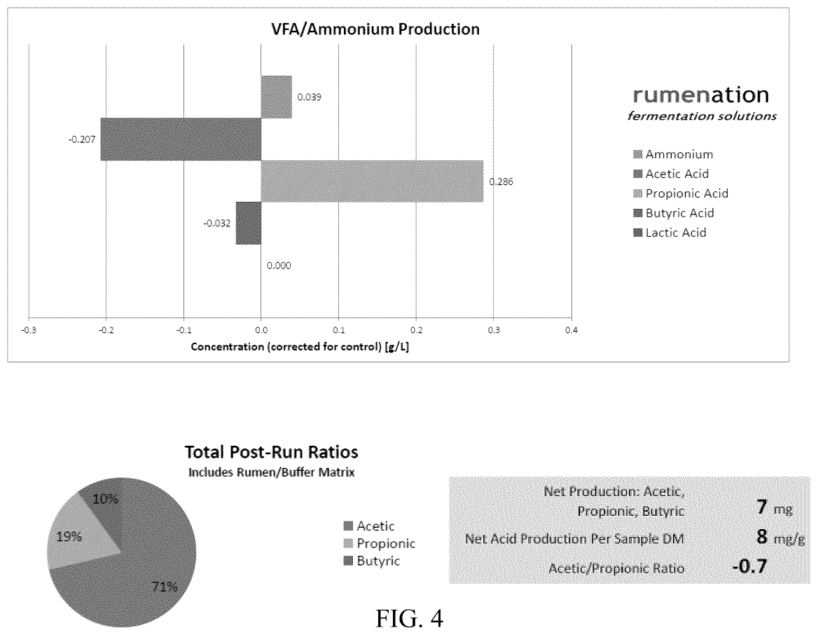

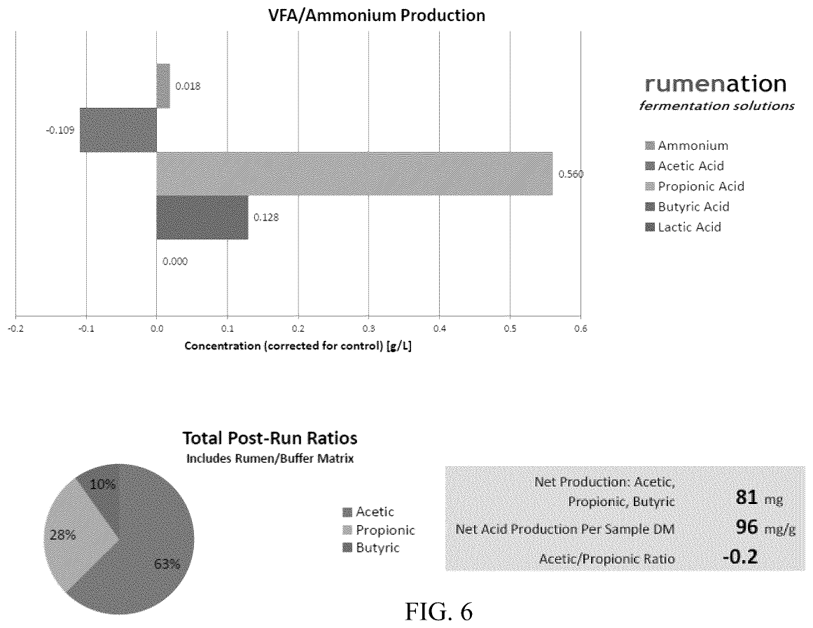

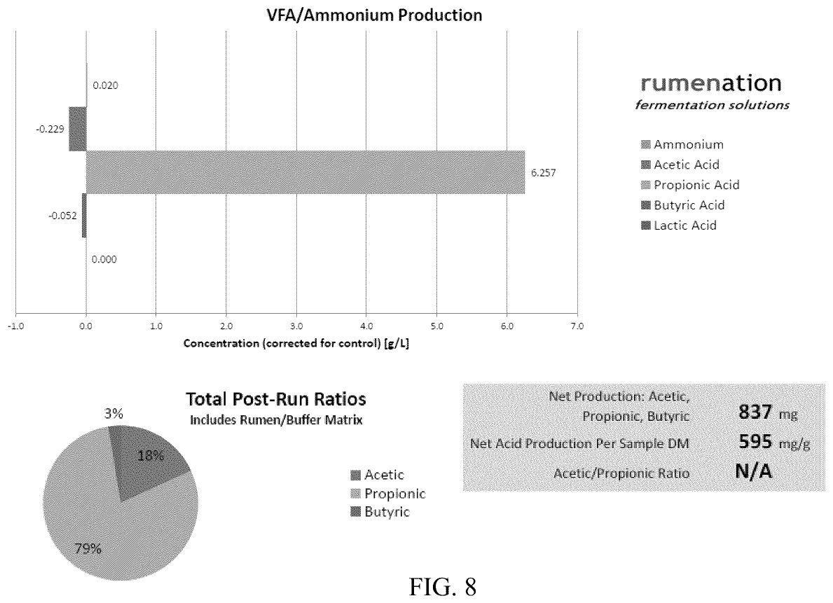

The following examples illustrate the efficacy of the disclosed composition in comparison to traditional methods of treating cows. Specifically, FIGS. 2, 3, 5, and 7 show the efficacy of propylene glycol in treating cows; FIGS. 4 and 6 show the efficacy of keto gel (another propylene glycol-based method of treatment which may be comprised of propylene glycol, vitamin A, D, and E, niacin, choline, methionine, and cobalt (vitamin B12 precursor)) in treating cows; and FIGS. 8 through 10 show the efficacy of the disclosed composition in treating cows. As clearly outlined in the examples, the disclosed composition is on the order of nine- to twenty-times (9-20×) more efficient at stimulating volatile fatty acid and ammonium production in the cow compared to either traditional method. Not only does the disclosed composition significantly outperform either propylene glycol or keto gel, the disclosed composition also achieves the surprising results in fewer dosage administrations to the cow. By decreasing the number of times the cow needs to be administered the disclosed composition, the overall stress of the cow is decreased and the overall wellbeing of the cow is increased.

### General Methodology

Dry-matter As Fed/As Tested. Dry-matter as fed (or “as Received”) is based on the feed or forage prior to drying or grinding. This value is taken from an already-run NIR report, or may be taken from an in-house NIR or actual oven dry-matter reading. It has no bearing on any other reported values, which are made on a dry-matter basis. The dry-matter as tested is the dry-matter portion of the 1-mm screened, dried and ground feed used for the analysis. If the sample did not require oven-drying, its grind is slightly drier than its as-fed value (such that an alfalfa hay received at 89.0% DM might actually test at 91.0% DM due to some drying on grinding). If the commodity was oven-dried, its DM as tested will of course be significantly higher than received. Most of the dried/ground feeds we test in vitro are around 94% DM, or 6% moisture.

Corrections for Control. The rumen fluid we use contains both intrinsic and purposefully added fermentable material (in addition to its microbial population), so that even a sample-free control jar of rumen fluid and buffer solution will generate typical fermentation products (gas pressure, new microbial biomass, and volatile fatty acids) and produce a drop in pH. As such, when these values are reported for the sample, they are corrected as the difference between the sample and the control, so that the sample contribution alone is reported. This also applies to gas data points throughout the run. Separate controls are used for bagged and unbagged runs due to the different rumen/buffer ratios we have optimized for each test type.

Normalization to Reference. Whereas control correction applies within the run, a “normalization” additionally helps provide useful run-to-run comparisons. This is necessary on account of day-to-day variations in rumen pH and microbial population densities and ratios. We maintain a “Reference TMR” as a rolling composite of high-performing TMRs. It is regularly replenished in small proportion, remixed, and kept dry. Maintaining a four-month rolling average of its performance measures, which allows adjustment for variations of rumen performance run-to-run. Sample values are reported both as actual measures (corrected for control), and also as normalized measures (corrected for control and also shifted to account for the performance of the Reference TMR). For example, if the Reference TMR produced 7% more gas on a run than the value of its four-month average, all samples'normalized gas values would be shifted downward by 7%. Similar normalizations are made digestion and microbial biomass production, and are reported in boldface and blue.

pH and pH Compared with Control. pH is of concern because rapid energy production of a feed may be accompanied by an excessive drop in pH, correlated with rumen acidosis. As such, the end-of-run pH value is reported, as well as the change in pH as compared with the control. The change in pH is therefore a measure of the degree to which the sample drove down the pH value.

Digestion and Nutritional Changes. Overall sample digestion is reported for bagged in vitro tests, as a positive percentage. This represents the consumption of sample on a dry-matter basis. For example: a 1.000 g sample at 95.00% dry-matter-as-tested was massed at 0.5788 g (after bag-weight subtraction) when dried-down thoroughly after the run. Therefore, its digested mass was 0.9500 g−0.5788 g=0.3712 g, and its digestion as a percent was therefore 0.3712 g÷0.9500 g=39.1%. Digestion is one of the values that is also reported as normalized, based on the performance of the Reference TMR. For unbagged analyses, a comparison is made between nutritional quantities before the run and after the run (by NIR analysis with correction for the control), along with before-and-after masses. Values are typically reported for Crude Protein, Degradable Protein, Starch, Crude Fat, NDF, and ADF. Values for these are reported as percent changes, not as digestions, as thus report as negative. For example an NDF digestion of 24.5% for the sample will report as −24.5%, which means that 24.5 percent of the sample's NDF was degraded during the run, relative to whatever amount the sample began with. Note that for protein changes, the value will be made less negative—or offset somewhat—by actual microbial protein production. (To get a true protein digestion value, consider the in situ analysis, which uses sample bagging as well as post-run nutritional analysis.)

Gas Production. Methane (CH₄) and carbon dioxide (CO₂) gases are the primary gas products of ruminal fermentation. Volumes are reported in milliliters (mL) and represent the sum of all gases produced in excess of the control. Volumes are presented graphically as a production trend throughout the run. Note that gases, while being produced, may also be utilized by the bugs or dissolved in the fluids, and thus “gas production” is the net value of production less these smaller effects. Volumes beyond 24 hours or 5 hours may be shown on the graph, but the 24.0-hr. or 5.0-hr. point of volume is reported in the table and used for most end-point calculations. These values are also reported as normalized based on the performance of the Reference TMR in the same measure.

Gas Production per Digested Dry Matter. On bagged samples, the report states the volume of gas that is produced per mass digested. For example: A 1.000 g hay sample at 90% dry matter produced 60.0 mL of gas (above the control) by the end of the run, and showed a 35.0% digestion, dry-matter basis. Thus, its actual digested matter was 35% of 0.900 g, or 0.315 g. Correspondingly, its Gas Production per Digested Dry Matter was 60.0 mL÷0.315 g=190 mL/g. A sample with low digestion and moderate gas production may still produce a relatively high value by this measure.

CO₂ Fraction. The CO₂ fraction is reported as the partial pressure and volume portion of CO2 as compared to total gases, to a precision of 1 point, such as 43%. It can be assumed that the remaining gas is primarily methane (CH4) with an undetermined small portion of hydrogen (H2). Nitrogen exists to some degree in an actual cow's rumen cavity due to swallowing of air, but is not present in vitro.

Fast/Slow Pool Gas Pools. The fast pool represents gas production due primarily to starch and soluble fiber, and the slow pool represents gas production due primarily to insoluble available fiber. Although interpreted from a mathematical model, they are tied to the total gas data, and at any time point the value of the two pools will sum to the actual gas total. The result is presented graphically, and also as an end-of-run (24.0 hr.) ratio such as 54:46, along with two key time points: The first is the point in time in which the fast pool reaches 90% of its projected maximum gas production. The slow pool gas curve typically provides a tell-tale inflection point, at maximum rate of gas production. This point in time is also reported.

Digestion Rates. These rates are the instantaneous rates of digestion, in percent digestion per hour, due to each energy pool at the time point of each's max rate. For example, a slow pool digestion rate of 2.35 kd/hr at a slow pool max time of 12.5 hours indicates that, at this instant of time during the run, the sample was being digested at a rate of 2.35% per hour (were that rate to continue), due to the slow pool. Since the fast pool curve continues upward slightly even after flattening out somewhat, the total digestion rate of the sample at the slow-pool max time point is slightly greater than the slow pool rate alone. By contrast, the reported fast-pool rate is typically at or near the onset of fermentation, and thus represents the rate of the whole sample at that instant. Toward the end of the run, most of the sample's digestion rate results from the slow-pool energy.

Microbial Biomass Production. When a bagged sample is removed after the run, the remaining fluids contain digested rumen material along with an increase in rumen microbial mass due to growth and reproduction of microbes during the run. This mass increase is primarily microbial protein (a protein source for the animal beyond the feed's direct protein), a strong performance measure for a sample. As with gas production, each sample's biomass production is control-corrected, measuring the difference between its microbial mass and the control's microbial mass simply due to the rumen fluid. This value, in milligrams, is also shifted based on the performance of the Reference TMR in the same measure.

Microbial Biomass per Digested Matter. As with gas production, the microbial biomass production is reported both as a standalone value and as a rate per mass digested. For example: A 1.000 g corn silage sample at 94.0% dry matter (as tested) produces 47 mg of microbial biomass (above the control) at the end of the run, and shows a 55% digestion. Thus, its actual digested matter is 55% of 0.940 g, or 0.517 g. Correspondingly, its Biomass Production per Digested Mass is 47 mg÷0.517 g=91 mg/g. Thus, a sample with high digestion and moderate biomass production may show a low value by this measure.

In Vitro Measured Feed Value Score (IFV). This score provides a performance prediction in addition to the traditional RFV and newer RFQ. Numbers fall in the same range as RFV or RFQ, such as a low value of 110 or a high value of 210. When the IFV differs by 15 or more points than the NIR-predicted RFQ, it suggests that the hay will under-or over-perform its RFQ value due to factors that RFQ or RFV have not accounted for. This can be profitable information for those buying and selling hay or add confidence to nutritionists'feed rationing.

In Vitro Relative Performance Score (IRP). This can be useful run-to-run, but is preferred for ranking sample performances for two or more samples on the same run. A score of 10.0 indicates a performance equal to the Reference TMR, and values may be reported either higher or lower than 10.0.

Volatile Fatty Acid (VFA) and Ammonium Production. VFA and ammonium production are reported in grams per liter, above the control. (As 140 mL of fluids are used for in vitro analyses, this means that the actual sample production is 0.14× this value, in grams.) Colored bar graphs provide a visualization of their production relative to each other. Total production of the acetic, propionic, and butyric components is tallied in milligrams, and also presented in milligrams per original sample dry-matter. For example, net acid production of 170 mg caused by an unbagged sample (1.500 g) at 92% moisture would be reported as 170 mg÷(0.92×1.500 g)=170 mg÷1.353 g=126 mg/g. The acetic/propionic acid ratio is of particular interest and is reported alongside these other totals. Lastly, a pie chart is presented to show the absolute acetic, propionic, and butyric proportions in the sample jar, combining the total effects of the sample/rumen/buffer matrix.

Fatty Acid (FA) Production. Rather than to analyze post-run fluids as with VFAs, the post run solids are dried and ether-extracted, then compared to a full pre-run ether-extract/fatty-acid profile via HPLC analysis. If arranged, these results require an additional 48 hrs.

### In Vivo Methodology

Each Example below was conducted in vivo by submerging a sample containing propylene glycol, Keto-Gel, or the disclosed composition in rumen fluid from a cow. The in vivo testing was done according to the following methodology and parameters.

Basic Run Parameters. A 30-jar Ankom RF Gas Production System is utilized, allocating 2-8 jars for controls and references, and occasionally additional jars for regular in-house research, allowing remaining jars to be available for clients'sample analyses. During our standard run of just over 24 hours, cumulative gas pressure data is sent wirelessly to a computer for recording, every two minutes during the first 2-5 hours, and every five minutes thereafter. Samples were first dried at 600° C. and ground through a 1.0 mm screen. If bagged, 1.000 g is measured into a non-digestible micro-pore bag, heat-sealed and weighted before placing into the jar. If unbagged, 1.500 g is measured into the jar directly. Samples are incubated in a blend of freshly primed rumen fluid and Kansas State Buffer solution (pH 6.80), with jars resting in a 390° C. water bath while being agitated gently at 40 cycles per minute. Jar headspace is purged with CO2 prior to the run.

Rumen Fluid Preparation. Rumen fluid is prepared from freshly collected rumen material from one or two cannulated cows for its microbial populations. The preparation process involves blending the raw material in a food processor, expressing the fluid in stages by use of a press, and priming the fluid with nutrient, CO₂ purge, and temperature-controlled water bath. A cheesecloth filtration is used to provide homogeneity of the fluid, free of any visible plant material or nutrient clumping. By the time the 30-60 minute priming process is completed, the rumen fluid is back to typical rumen temperature and its “bug” population is diverse and active. The fluid is vigorously re-suspended prior to taking a measure for each sample (using a customized volumetric vessel), and sample jars with buffer receive a precise amount (test type specific) within a narrow time window.

pH. An Orion temperature-correcting pH meter is used, with precision to two decimal places, calibrated regularly with fresh standards. Measurements are taken of rumen throughout preparations stages, controls at run start, and all controls, reference samples, and client samples at the end of the run.

Digestion. For bagged analyses, the digested portion is measured by comparing the dry-matter basis sample mass before and after the run. The pre-run mass is 1.000 g multiplied by the as-tested dry-matter percentage, obtained on the powder at 1350° C. for 2 hrs. or NIR-predicted equivalent. The post-run mass is the mass of the 1050° C., 3+ hr. dried bagged sample, less the originally determined bag mass.

Gas Production. As mentioned, jar pressure data is recorded throughout the run. Gas is being produced, causing the jar's fixed headspace to become increasingly pressurized during the run. These pressure readings are converted to the volume equivalence that would be taken up by the combined gases in the jar's headspace if at standard atmospheric pressure.

Microbial Biomass Production. For bagged analyses, the actual microbial biomass production (primarily microbial protein) is collected by removing the bagged feed and centrifuging the remaining fluids of each sample jar in two 70 mL tubes. The pellets are swabbed from the centrifuge tube, spread out, and dried down at 1050° C. for 3+ hrs. Microbial biomass production of the control is also measured, and the difference between the sample's biomass and control's biomass is regarded as the microbial biomass produced during the run.

Nutritional Analysis of Solids. For unbagged analyses, solids are centrifuged and collected similarly, but instead include a post-run blend of both fluids and sample residual. This is dried down at 600° C. for 4-6 hours, pestled to a powder for packing into an NIR cell, and run for NIR analysis (included in the cost of the test and not reported). Absolute nutritional values are calculated from NIR predictions (with in-house correction methods for the unique properties of post-run solids), the NIR dry-matter reading, and the actual dry-down mass. Subtracted from these are the same components measured as the average of two control jars (doubled up to provide enough rumen fluid solids for the NIR analysis).

Fast/Slow Gas Pools. The fast-and slow-pool graphs are generated by a proprietary mathematical method using gas volume data from the actual run. Key time points and digestion rates are tied to these initial calculations and curves.

CO₂ Fraction. The post-run CO2 fraction is estimated by a proprietary method involving mixing and extracting an aliquot of the gas from the jar headspace, reacting this gas with a weakly basic solution, and measuring the pH shift of that solution against calibrations and controls specific to that run.

VFA/Ammonium Production. These soluble components are measured on the post-run supernatant fluid after centrifuging. Ammonium is measured by ion-specific electron (ISE) probe with ion strength adjustment (ISA), and the four VFAs (acetic, propionic, butyric, and lactic acids) are measured on a vacuum-filtered aliquot by high performance liquid chromatography (HPLC) using a ligand-exchange column and UV detection.

### In Situ Methodology

In some embodiments, testing may be conducted in situ by administering a sample containing propylene glycol, Keto-Gel, or the disclosed composition to a cow. The in situ testing may be done according to the following methodology and parameters.

Normalization to Reference is not practical to perform in situ, and thus side-by-side sample comparisons are recommended even more strongly for in situ analyses.

Basic Run Parameters. We mix and select two portions from the original sample that will dry down to around 30-40 grams each. The first portion, if not already a dry commodity, is dried at 600° C., massed, and transferred into a non-digesting flow-through fiber bag, heat-sealed to contain the sample. (On wet samples, the entire wet subsample is bagged after drying to avoid particle sorting issues that would arise with subsampling dried unground feeds.) The sample is then tethered 12-18 inches into the rumen cavity of a cannulated cow. The second portion is dried at 600° C., massed, dried further at 1050° C., and massed finally for a dry matter correction on the pre-run sample. Sometime near 7.0 hours, the in situ sample is removed from the cow, cold water rinsed, and oven-dried either at 1050° C. (for digestion-only analyses) or at 600° C. and ground through a 1 mm screen (for full analyses requiring NIR). Oven dry-downs are a minimum of 6 hours, and the post-run sample is broken up during the dry-down to facilitate drying, as it is saturated at post-run and may tend to form a crust that inhibits complete dry down.

Duration. This represents the actual time that the sample was submerged in the cow's rumen cavity. 7.0 hours is the target, and final reported values are approximately linearly for runs slight shorter or longer.

Dry-matter As Fed/As Tested. Dry-matter as fed is outlined above. The dry-matter as tested is the dry-matter percentage of the 600° C. oven-dried sample bagged and placed in situ. If the sample is not “wet” and thus oven-dried prior to testing (such as for a hay, grain, or corn product), the two dry-matters will report the same. Most forages and TMRs we test in situ at about 96% DM, or 4% moisture.

Digestion. For digestion-only analyses, true total digestion is calculated and reported as above on the 24-hr. bagged samples, but with a larger sample (30-40 g dry-matter) and with the difference that it typically involves particle-size as-is (no grinding). For full analyses including nutritional digestions, the total digestion is calculated using DM-correction from the NIR analysis, as the dry-down is at 600° C. and not 1050° C.

Gas production is not measured in situ.

Nutritional Percent Changes. These are calculated and reported as above with unbagged in vitro, but as the post-run tested portion was bagged, it does not include rumen nor require control correction. Furthermore, the sample's protein changes can be accurately measured in situ, because microbial protein growth does not offset the result as it does in the unbagged analyses.

### EXAMPLES

Each Example below was conducted in vivo by submerging a sample containing propylene glycol, Keto-Gel, or the disclosed composition in rumen fluid from a cow. The in vivo testing was done according to the in vivo methodology outlined previously.

Example 1. FIG. 2 shows in vivo production of volatile fatty acids and ammonium resulting from submergence of a sample containing propylene glycol in rumen fluid from a cow for about 5 hours. A 1.50 g sample of ground powder containing propylene glycol was used. The jar fluid was centrifuged and the supernatant probed with ammonium ISE. VFAs were measured and identified via HPLC using filtered aliquots. Sample production values have been corrected for control production. As seen, propionic acid was produced at a concentration of 0.275 g/L, corrected for control; acetic acid was produced at a concentration of 0.095 g/L, corrected for control; lactic acid was produced at a concentration of 0.043 g/L, corrected for control; ammonium was produced at a concentration of 0.013 g/L, corrected for control; and butyric acid experienced a decrease in production, amounting to a concentration of −0.019 g/L, corrected for control. The total ratio of acetic acid:propionic acid:butyric acid was 3.7:1.9:1.

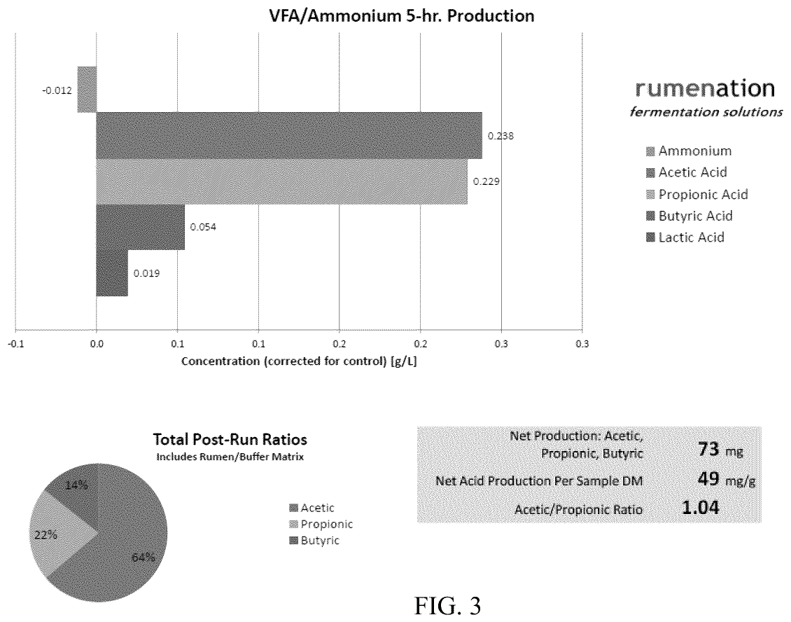

Example 2. FIG. 3 shows in vivo production of volatile fatty acids and ammonium resulting from submergence of a sample containing propylene glycol in rumen fluid from a cow four about 5 hours. A 1.50 g sample of ground powder containing propylene glycol was used. The jar fluid was centrifuged and the supernatant probed with ammonium ISE. VFAs were measured and identified via HPLC using filtered aliquots. Sample production values have been corrected for control production. As seen, propionic acid was produced at a concentration of 0.229 g/L, corrected for control; acetic acid was produced at a concentration of 0.238 g/L, corrected for control; lactic acid was produced at a concentration of 0.019 g/L, corrected for control; butyric acid was produced at a concentration of 0.054 g/L, corrected for control; and ammonium experienced a decrease in production, amounting to a concentration of −0.012 g/L, corrected for control. The total ratio of acetic acid:propionic acid:butyric acid was 4.6:1.6:1.

Example 3. FIG. 4 shows in vivo production of volatile fatty acids and ammonium resulting from submergence of a sample containing keto gel in rumen fluid from a cow for about 24 hours. The jar fluid containing the sample was centrifuged and the supernatant probed with ammonium ISE. VFAs were measured and identified via HPLC using filtered aliquots. Sample production values have been corrected for control production. As seen, propionic acid was produced at a concentration of 0.286 g/L, corrected for control; no lactic acid was produced; acetic acid experienced a decrease in production, amounting to a concentration of −0.207 g/L, corrected for control; butyric acid experienced a decrease in production, amounting to a concentration of −0.032 g/L, corrected for control; and ammonium was produced at a concentration of 0.039 g/L, corrected for control. The total ratio of acetic acid:propionic acid:butyric acid was 7.1:1.9:1.

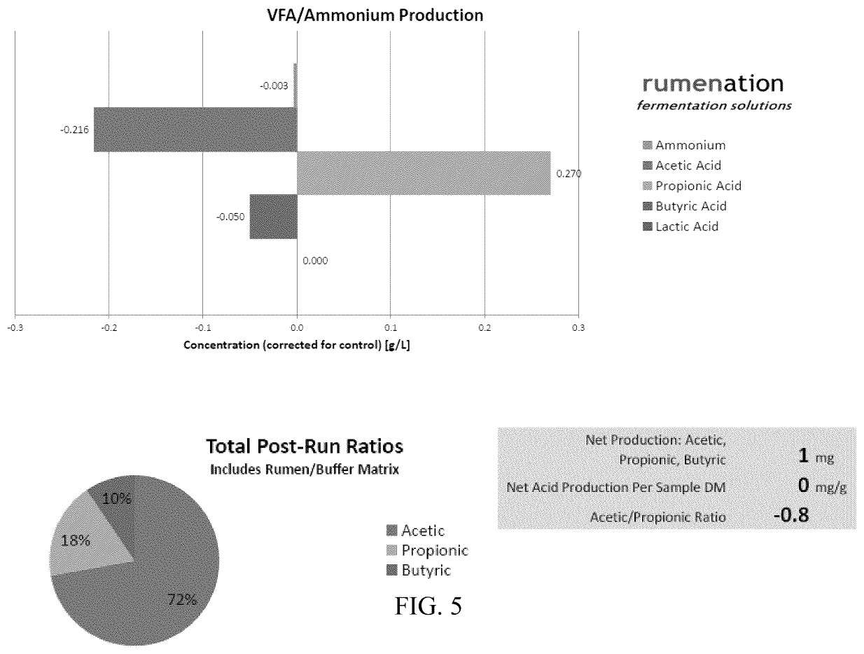

Example 4. FIG. 5 shows in vivo production of volatile fatty acids and ammonium resulting from submergence of a sample containing propylene glycol in rumen fluid from a cow four about 24 hours. The jar fluid containing the sample was centrifuged and the supernatant probed with ammonium ISE. VFAs were measured and identified via HPLC using filtered aliquots. Sample production values have been corrected for control production. As seen, propionic acid was produced at a concentration of 0.270 g/L, corrected for control; no lactic acid was produced; acetic acid experienced a decrease in production, amounting to a concentration of −0.216 g/L, corrected for control; butyric acid experienced a decrease in production, amounting to a concentration of −0.050 g/L, corrected for control; and ammonium experienced a decrease in production, amounting to a concentration of −0.003 g/L, corrected for control. The total ratio of acetic acid:propionic acid:butyric acid was 7.2:1.8:1.

Example 5. FIG. 6 shows in vivo production of volatile fatty acids and ammonium resulting from submergence of a sample containing keto gel in rumen fluid from a cow for about 24 hours. The jar fluid containing the sample was centrifuged and the supernatant probed with ammonium ISE. VFAs were measured and identified via HPLC using filtered aliquots. Sample production values have been corrected for control production. As seen, propionic acid was produced at a concentration of 0.560 g/L, corrected for control; no lactic acid was produced; acetic acid experienced a decrease in production, amounting to a concentration of −0.109 g/L, corrected for control; butyric acid was produced at a concentration of 0.128 g/L, corrected for control; and ammonium was produced at a concentration of 0.018 g/L, corrected for control. The total ratio of acetic acid:propionic acid:butyric acid was 6.3:2.8:1.

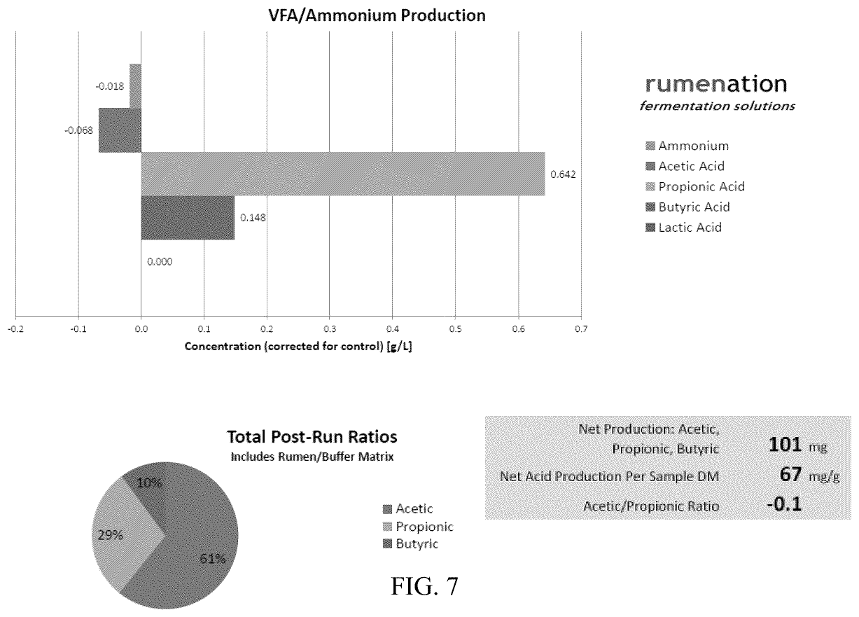

Example 6. FIG. 7 shows in vivo production of volatile fatty acids and ammonium resulting from submergence of a sample containing propylene glycol in rumen fluid from a cow four about 24 hours. The jar fluid containing the sample was centrifuged and the supernatant probed with ammonium ISE. VFAs were measured and identified via HPLC using filtered aliquots. Sample production values have been corrected for control production. As seen, propionic acid was produced at a concentration of 0.642 g/L, corrected for control; no lactic acid was produced; acetic acid experienced a decrease in production, amounting to a concentration of −0.068 g/L, corrected for control; butyric acid was produced at a concentration of 0.148 g/L, corrected for control; and ammonium experienced a decrease in production, amounting to a concentration of −0.018 g/L, corrected for control. The total ratio of acetic acid:propionic acid:butyric acid was 6.1:2.9:1.

Example 7. FIG. 8 shows in vivo production of volatile fatty acids and ammonium resulting from submergence of a sample containing the disclosed composition in rumen fluid from a cow four about 24 hours. An 84 g dry sample having 20% calcium by weight was submerged in a jar containing rumen fluid. The jar fluid containing the sample was centrifuged and the supernatant probed with ammonium ISE. VFAs were measured and identified via HPLC using filtered aliquots. Sample production values have been corrected for control production. As seen, propionic acid was produced at a concentration of 6.257 g/L, corrected for control. In comparison to Example 7, which had the highest concentration of propionic acid production by traditional methods, the concentration of propionic acid produced by the disclosed composition is about 10 times (10×) greater than that produced by administering propylene glycol to the cow. Ammonium was produced at a concentration of 0.020 g/L, corrected for control. No lactic acid was produced. Butryic acid experienced a decrease in production, resulting in a concentration of −0.052 g/L, corrected for control; and acetic acid also experienced a decrease in production, resulting in a concentration of −0.229 g/L, corrected for control. The total ratio of acetic acid:propionic acid:butyric acid was 6:26:1. As is clearly evident, administration of the disclosed composition allows the cow having received the composition to increase the production of propionic acid within the rumen. The use of calcium propionate aids in the reduction of the other acids and increase in the propionic acids. The high amounts of propionic acids relates to high energy production. Each sample of the rumen fluid may vary slightly in ingredients and thus the slightly different results of the acid production.

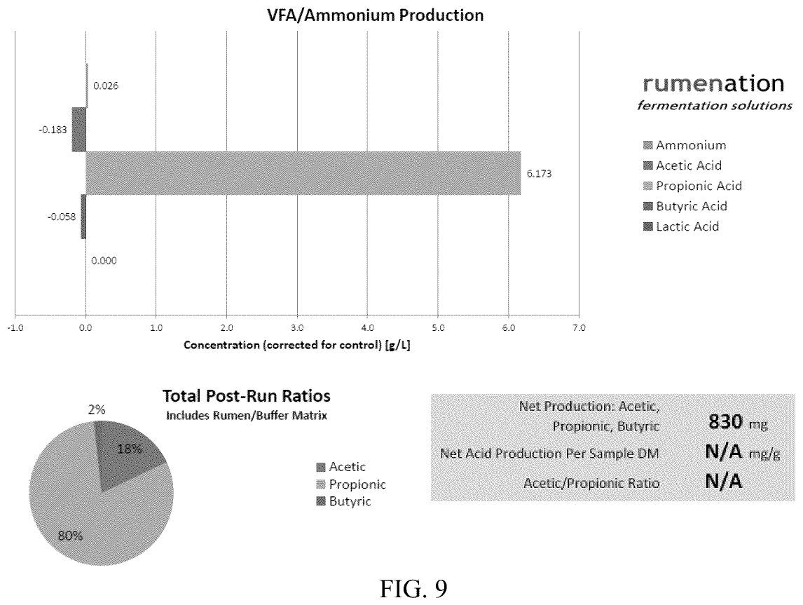

Example 8. FIG. 9 shows in vivo production of volatile fatty acids and ammonium resulting from submergence of a sample containing the disclosed composition in rumen fluid from a cow four about 24 hours. A 252 g dry sample of the disclosed composition was submerged in a jar containing rumen fluid. The jar fluid containing the sample was centrifuged and the supernatant probed with ammonium ISE. VFAs were measured and identified via HPLC using filtered aliquots. Sample production values have been corrected for control production. As seen, propionic acid was produced at a concentration of 6.173 g/L, corrected for control. In comparison to Example 7, which had the highest concentration of propionic acid production by traditional methods, the concentration of propionic acid produced by the disclosed composition is about 9 times (9×) greater than that produced by administering propylene glycol to the cow. Ammonium was produced at a concentration of 0.026 g/L, corrected for control. No lactic acid was produced. Butryic acid experienced a decrease in production, resulting in a concentration of −0.058 g/L, corrected for control; and acetic acid also experienced a decrease in production, resulting in a concentration of −0.183 g/L, corrected for control. The total ratio of acetic acid:propionic acid:butyric acid was 9:40:1. As is clearly evident, administration of the disclosed composition allows the cow having received the composition to increase the production of propionic acid within the rumen.

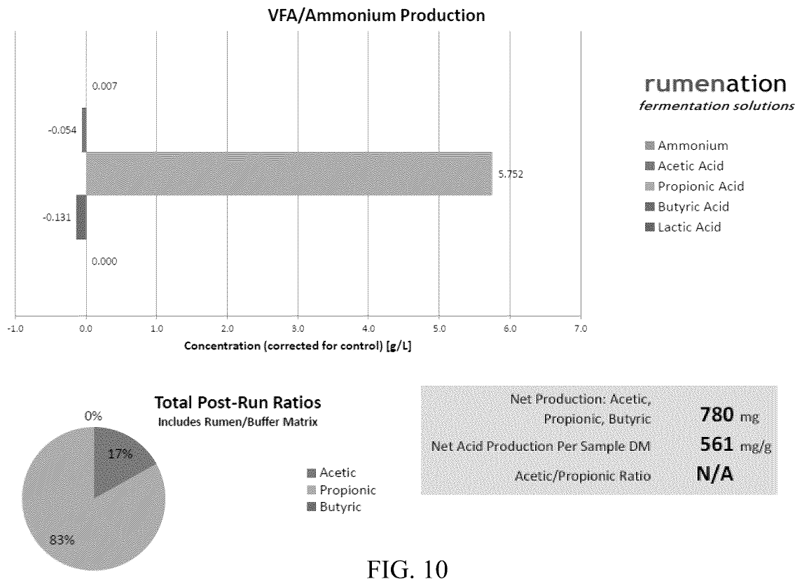

Example 9. FIG. 10 shows in vivo production of volatile fatty acids and ammonium resulting from submergence of a sample containing the disclosed composition herein in rumen fluid from a cow for about 24 hours. A 219 g dry sample of the disclosed composition, containing 64.45 g of calcium propionate, 5.45 g of probiotic, and 5.45 g of niacin, was placed in jar containing rumen fluid. The jar fluid containing the sample was centrifuged and the supernatant probed with ammonium ISE. VFAs were measured and identified via HPLC using filtered aliquots. Sample production values have been corrected for control production. As seen, propionic acid was produced at a concentration of 5.752 g/L, corrected for control. In comparison to Example 7, which had the highest concentration of propionic acid production by traditional methods, the concentration of propionic acid produced by the disclosed composition is about 9 times (9×) greater than that produced by administering propylene glycol to the cow. Ammonium was produced at a concentration of 0.007 g/L, corrected for control. No lactic acid was produced. Butryic acid experienced a decrease in production, resulting in a concentration of −0.131 g/L, corrected for control; and acetic acid also experienced a decrease in production, resulting in a concentration of −0.054 g/L, corrected for control. The total ratio of acetic acid:propionic acid:butyric acid was 1:4.9:0. As is clearly evident, administration of the disclosed composition allows the cow having received the composition to increase the production of propionic acid within the rumen.

### Additional Terms and Definitions

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to treating or addressing a particular condition for a cow (e.g., a fresh cow having just given birth to a calf); however, other conditions (e.g., disease, metabolic changes or challenges, pre-fresh cows, post-fresh cows, ketonic cows, etc.) are also contemplated.

In one embodiment, the terms “about” and “approximately” refer to numerical parameters within 10% of the indicated range. The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A composition for increasing calcium levels in an animal, the composition comprising:
calcium propionate provided in a concentration ranging from about 80% to about 88% by weight, the calcium propionate being both a glucose precursor and a source of calcium to increase calcium levels in the animal;
at least one probiotic component provided in a concentration ranging from about 6% to about 10% by weight; and
niacin provided in a concentration ranging from about 6% to about 10% by weight, the niacin being a glucose precursor to increase energy levels in the animal,
the composition increasing propionic acid production in the animal for at least 12 hours.

2. The composition of claim 1, wherein the concentration of calcium propionate provides a minimum of 44 grams of available calcium for uptake by the animal.

3. The composition of claim 1, wherein the concentration of the at least one probiotic component provides a minimum of 15 grams of available probiotic for uptake by the animal.

4. The composition of claim 1, wherein the concentration of calcium propionate provides a minimum of 185 grams of available glucose for uptake by the animal, wherein niacin facilitates the uptake.

5. The composition of claim 1, wherein the at least on probiotic component comprises a yeast.

6. The composition of claim 1, wherein the composition promotes biomass production by rumen-bugs in a rumen of a cow.

7. The composition of claim 1, wherein the composition increases production of propionic acid in the animal by at least 60%.

8. The composition of claim 1, wherein the concentration of niacin prevents ketosis in the animal.

9. A composition for administration to a cow, the composition comprising:
calcium propionate provided in a concentration of about 86% by weight, the calcium propionate being both a glucose precursor and a source of calcium to increase calcium levels in the cow;
at least one probiotic component provided in a concentration of about 7% by weight; and
niacin provided in a concentration of about 7% by weight, the niacin being a glucose precursor to increase energy levels in the cow.

10. The composition of claim 9, wherein calcium propionate and niacin are the only sources of glucose precursors in the composition.

11. The composition of claim 9, wherein the at least on probiotic component comprises a yeast.

12. A method of increasing energy and calcium levels in a cow, the method comprising:
preparing a composition of at least one bolus to administer to a cow comprising:
measuring calcium propionate provided in a concentration ranging from about 80% to about 88% by weight; at least one probiotic component provided in a concentration ranging from about 6% to about 10% by weight; and niacin provided in a concentration ranging from about 6% to about 10% by weight;
mixing the calcium propionate, at least one probiotic, and niacin for at least three minutes;
placing the mixed calcium propionate, at least one probiotic, and niacin into a dissolvable gel cap;
administering the composition in the at least one bolus to a cow for increasing energy and calcium levels in the cow.

13. The method of claim 12, wherein administering to the cow a therapeutically effect amount of the composition comprises:
loading the therapeutically effective amount of the composition into a bolus gun; and
depositing the bolus onto a back of a tongue of the cow, such that the cow may swallow the bolus for digestion.

14. The method of claim 12, wherein administering to the cow a therapeutically effect amount of a composition comprises administering a single dose of the composition to the cow.

15. The method of claim 14, wherein a single dose comprises three boluses.

16. The method of claim 12, wherein administering to the cow a therapeutically effect amount of a composition comprises:
administering a first dose of the composition to the cow at time one; and
administering a second dose of the composition to the cow at time two, time two being later than/after time one.

17. The method of claim 12, wherein the composition is administered to a fresh cow having given birth to a calf.

18. The method of claim 12, wherein the composition is administered to a pre-fresh, pregnant cow.

19. The method of claim 12, wherein the composition is administered to a ketonic cow.
